(19) United States
(12) Patent Application Publication
(10) Pub. No.: US 2016/0228289 A1
(43) Pub. Date: Aug. 11, 2016

(54) COLD THERAPY DEVICE WITH THERMO-MECHANICAL MIXING VALVE

(71) Applicant: Jeffrey T. Mason, Escondido, CA (US)

(72) Inventor: Jeffrey T. Mason, Escondido, CA (US)

(21) Appl. No.: 14/619,955

(22) Filed: Feb. 11, 2015

(51) Int. Cl. A61F 7/10

(52) U.S. Cl. CPC A61F 7/10; A61F 2007/0056

(57) ABSTRACT

A cold therapy unit incorporates a cold reservoir containing a cold treatment fluid, typically water with ice and a treatment pad with an inlet line and an outlet line. A thermo-mechanical mixing valve receives treatment fluid from the reservoir and warmer fluid from the pad in a ratio that results in treatment fluid of the desired temperature. A pump delivers that treatment fluid to the inlet line on the pad. A portion of the treatment fluid exiting the pad through an outlet line will return to the reservoir and a portion to the mixing valve as required to maintain a predetermined temperature in the mixing valve.

### BACKGROUND OF THE INVENTION

1. Field of Invention

The present invention relates generally to cold therapy, and specifically to improving the safety and effectiveness of a cold therapy unit which reduces pain and swelling at an injury site of a patient.

2. Description of Related Art

Cold therapy or cryotherapy (e.g., ice) is used to reduce pain and swelling, otherwise known as edema formation, from an acute injury or post-surgery site. The therapy is especially useful for injuries such as sprains, strains, pulled muscles, and pulled ligaments during sports and other activities. Cold therapy is also often recommended by orthopedic surgeons following surgery, as ice is one of the principles of Rest, Ice, Compression, and Elevation (RICE) therapy.

The ice principle, known as cryotherapy, is the use of cold or ice to lower the temperature of the injured tissue, which reduces the tissue's metabolic rate and helps the tissue survive the period following the injury. In a therapeutic setting, cryotherapy has become one of the most common treatments in orthopedic medicine.

Alternatives to ice include cold wraps such as re-freezable gel packs, which are less messy and reusable. Another popular alternative is portable cold therapy units, which are an effective and convenient alternative to icing, as the cold therapy unit circulates ice water through a treatment pad wrapped around an injury or surgery site of the patient to reduce pain and swelling. Cold therapy unit are often prescribed by doctors or selected by patients after surgery for use in their home.

According to the medical community's guidelines, the length of time that cold can be applied to an injury or wound site depends on the temperature of the treatment medium. For example, ice, which will have a surface temperature of 32 degrees F., should generally be limited to a maximum of 15-20 minutes at a time and no more frequently then every two hours. Exceeding these established limitations puts the patient at risk for further injury. For example, exceeding the 20 minute treatment limitation can cause frostbite or other damage to skin, tissue, and nerves. However a cold therapy unit, with a treatment pad having a surface temperature greater than 45 degrees F., can be applied continuously for as long as needed to reduce pain and swelling.

The portable cold therapy units existing in the prior art consist of a reservoir which hold ice and water, a pump to circulate the water and a treatment pad through which the water is circulated. All of the units that are intended for continuous use, (greater than 20 minutes), rely on body heat from the patient to warm the water from just above freezing to a safe level. This is typically done by controlling how fast the water flows through the pad, typically about 4 oz. per minute. Unfortunately there are many variables other than flow rate that effect the resulting temperature of the treatment water such as the size of the treatment pad, the amount of blood circulation the patient has at the treatment site and the location of the treatment site on the patient. For example an ankle typically has poor circulation while a shoulder has very good circulation. Also an ankle treatment pad is typically much smaller than a shoulder pad therefor the water would remain in the ankle pad for a very short time not allowing much time to warm up while it would remain in the shoulder pad much longer therefor warming to a much greater extent. The result is that the ankle is exposed to temperatures lower than what are safe and the shoulder doesn't see low enough temperatures to help much in the treatment.

The manufacturers of prior art devices deal with this issue in a couple of ways. One way is to put a thermometer in the line returning from the treatment pad to the reservoir and provide controls for the patient or medical practitioner to control the flow rate of the treatment fluid so that the desired temperature can be maintained. This method has some major drawbacks. One drawback is that the heath care practitioner as well as the patient need to fully understand the proper use of the device as well as the risks if not used according to directions. Unfortunately, the patient often does not read through the instructions and warnings and sometimes concludes that if a little is good a lot is better which often results in serious skin or nerve damage. Since the treatment area is numb from the cold the patient does not feel any pain from the damage that is occurring.

Another drawback to manually controlling temperature is that it sometimes results in ineffective treatment. This is because one of the major influences on the amount of heat that is transferred from the body to the treatment pad is the thickness and number of layers of dressing between the treatment pad and the skin. What happens is that a lot of dressing equals a lot of insulation resulting in little heat transfer which results in the thermometer showing that the water is too cold. The patient, according to the instructions, would decrease the flow resulting in a warmer treatment pad and thermometer reading. With that much insulation, in order to get enough heat transfer for the treatment to be affective, the patient would actually need to increase the flow rate rather than decrease resulting in a cooler treatment pad.

Another way that manufacturers of prior art cold therapy units deal with the problem of different treatment sites and different sizes treatment pads having different heat transfer rates, is by having a different fixed flow rate for each type of treatment pad. For example, an ankle pad might have a flow rate one quarter of that of a shoulder pad. While this method is safer than the manual control method, since the patient or medical practitioner cannot adjust the flow rate, it still does not address the issue of patients having differing blood circulation at any given treatment site due to factors such as age, health, smoking, prior surgeries at that location, etc.

There is another major problem with all of prior art portable units that rely on the water warming from just above freezing to a safe level in a single pass through the pad. The water enters the inlet port of the pad at just above freezing, travels an arduous path around barriers that prevent the water from shortcutting from the inlet directly to the outlet and then the water exits the outlet port at approximately 50 degrees F. What this means is that the inlet quadrant of the pad will be close to freezing while the outlet quadrant will be about 15 degrees warmer. This could result in skin or nerve damage localized at the inlet quadrant of the pad.

Many of the injuries such as skin necrosis, blistering and nerve damage, observed in patients using cold therapy units, are also observed in patients who did not use cold therapy. This and the fact that there is no way to determine what temperature the treatment site experienced, makes it very difficult if not impossible to determine the exact cause of the injury. This confusion has led to each manufacturer spending millions of dollars each year between litigation and injury compensation for cases that may or may not been a result of their product.

It is therefore advantageous to have a portable cold therapy unit that delivers a treatment fluid to a treatment pad entering the pad at consistent and safe temperature. It is also desirable that the treatment fluid circulates at a relatively high flow rate so that the temperature of the fluid exiting the pad is only slightly warmer than the fluid entering the pad. Such a unit would not be subject to miss-adjustment, cold spots on the pad, and in the event of litigation, testing of the unit to determine if it was maintaining a safe temperature is more easily accomplished.

### SUMMARY OF THE INVENTION

Embodiments of the present invention provide a cold therapy unit incorporating a cold reservoir containing a cold treatment fluid, typically water with ice and a treatment pad with an inlet line and an outlet line. A thermo-mechanical mixing valve receives treatment fluid from the reservoir and warmer fluid from the pad in a ratio that results in treatment fluid of the desired temperature. A pump delivers that treatment fluid to the inlet line on the pad. A portion of the treatment fluid exiting the pad through an outlet line will return to the reservoir and a portion to the mixing valve as required to maintain a predetermined temperature in the mixing valve.

In one embodiment of the present invention the mixing valve employs a sealed chamber with two inlet ports on opposite sides of the chamber, the first inlet port receiving cold fluid from the reservoir and the second inlet port receiving warmer fluid from the outlet line of the treatment pad. A bimetal strip reactive by bending in accordance with exposure temperature is oriented in the chamber to block off a first one of the two inlet ports when the temperature of the fluid in the chamber is below a predetermined lower threshold or the second one of the two inlet ports when the treatment fluid in the chamber is above a predetermined upper threshold. Adjustment screws calibrate the temperature at which the fluid in the chamber is maintained.

In another embodiment of the present invention the bimetal mixing valve employs a sealed chamber with two inlet ports on opposite sides of the chamber, the first inlet port receiving cold fluid from the reservoir and the second inlet port receiving warmer fluid from the outlet line of the treatment pad. A dual ended tapered needle valve is adapted for one end to be received in each of the inlet ports to gradually increase or decrease the flow of fluid as the needle valve is retracted from or inserted into each of the ports. A bimetal strip which is reactive in accordance with exposure temperature is oriented in the chamber to act on the dual ended needle valve causing the respective ends to be inserted or retracted from each of the ports depending on the temperature of the fluid in the chamber causing a restriction of flow in the first inlet port when the fluid in the chamber is below a predetermined threshold temperature or in the second inlet port when the treatment fluid in the chamber is above a predetermined threshold temperature. Adjustment screws are provided to calibrate the temperature at which the fluid in the chamber is maintained.

In another embodiment of the present invention the mixing valve employs a sealed chamber with two inlet ports on opposite sides of the chamber. A first inlet port receives cold fluid from the reservoir and a second inlet port receives warmer fluid from the outlet line of the treatment pad. A bimetal coil which is reactive by winding or unwinding in accordance with exposure temperature is oriented in the chamber to block off the first inlet port when the fluid in the chamber is below a predetermined threshold temperature or the second inlet port when the treatment fluid in the chamber is above a predetermined threshold temperature. An adjustment screw at the center of the bimetal coil is employed to calibrate the temperature at which the fluid in the chamber is maintained.

In another embodiment of the present invention the bimetal mixing valve employs a sealed chamber with two inlet ports on opposite sides of the chamber, a first inlet port receiving cold fluid from the reservoir and a second inlet port receiving warmer fluid from the outlet line of the treatment pad. A dual ended tapered needle valve is adapted for one end to be received in each of the inlet ports to gradually increase or decrease the flow of fluid as the needle valve is retracted from or inserted into each of the ports. A bimetal coil which is reactive by winding or unwinding in accordance with exposure temperature is oriented in the chamber to act on the dual ended needle valve causing the respective ends to be inserted or retracted from each of the ports depending on the temperature of the fluid in the chamber causing a restriction of flow in the first inlet port when the fluid in the chamber is below a predetermined threshold temperature or in the second inlet port when the treatment fluid in the chamber is above a predetermined threshold temperature. An adjustment screw at the center of the bimetal coil is employed to calibrate the temperature at which the fluid in the chamber is maintained.

The features, functions, and advantages that have been discussed can be achieved independently in various embodiments of the present disclosure or may be combined in yet other embodiments further details of which can be seen with reference to the following description and drawings.

### BRIEF DESCRIPTION OF THE DRAWINGS

FIG. 1A is a schematic representation of the elements of an exemplary embodiment;

FIG. 1B is a schematic representation of the elements of a second exemplary embodiment;

FIG. 2A is a section view of a first example bimetal valve for use in the exemplary embodiments in a first position;

FIG. 2B is a section view of the first example bimetal valve for use in the exemplary embodiments in a second position;

FIG. 3A is a section view of a second example bimetal valve for use in the exemplary embodiments in a first position;

FIG. 3B is a section view of the second example bimetal valve for use in the exemplary embodiments in a second position;

FIG. 4A is a section view of a third example bimetal valve for use in the exemplary embodiments in a first position;

FIG. 4B is a section view of the third example bimetal valve for use in the exemplary embodiments in a second position;

FIG. 5A is a section view of a fourth example bimetal valve for use in the exemplary embodiments in a first position;

FIG. 5B is a section view of the fourth example bimetal valve for use in the exemplary embodiments in a second position;

FIG. 6A is a section view of an alternative pressure valve for use in the exemplary embodiments in a first position;

FIG. 6B is a section view of the pressure valve for use in the exemplary embodiments in a second position;

FIG. 7A is a section view of a second example of the pressure valve for use in the exemplary embodiments in a first position;

FIG. 7B is a section view of the second example pressure valve for use in the exemplary embodiments in a second position; and,

FIG. 8 is a flow chart of a method for providing cooled water to a therapy pad employing the embodiment disclosed.

### DETAILED DESCRIPTION

Embodiments disclosed herein provide a novel method and apparatus for maintaining a safe temperature in ice water cooled cold therapy pads using a thermo-mechanical valve for flow mixing of pumped water to the pad by receiving return flow from the pad and a cold reservoir with ice water supply. In one embodiment the return flow from the pad is routed through a holding reservoir before being routed to the thermo-mechanical valve.

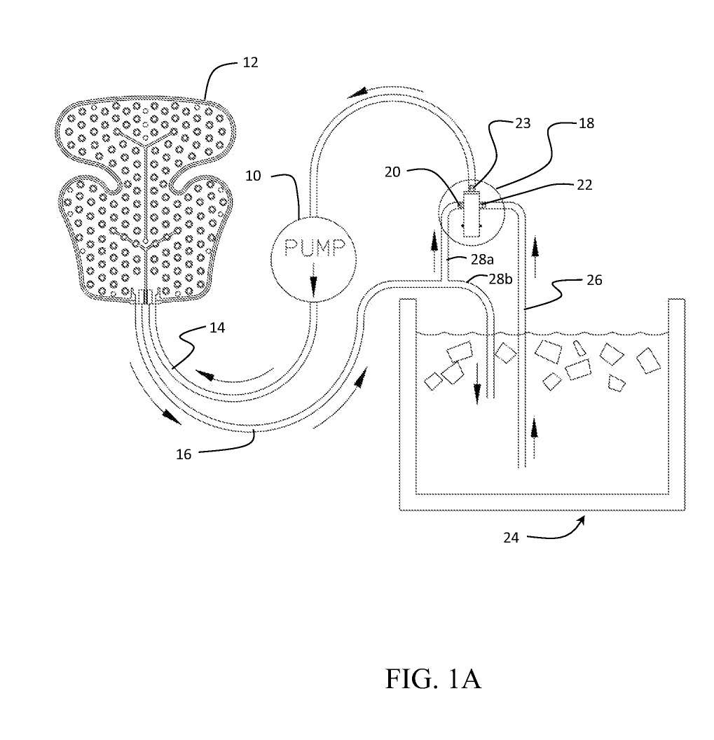

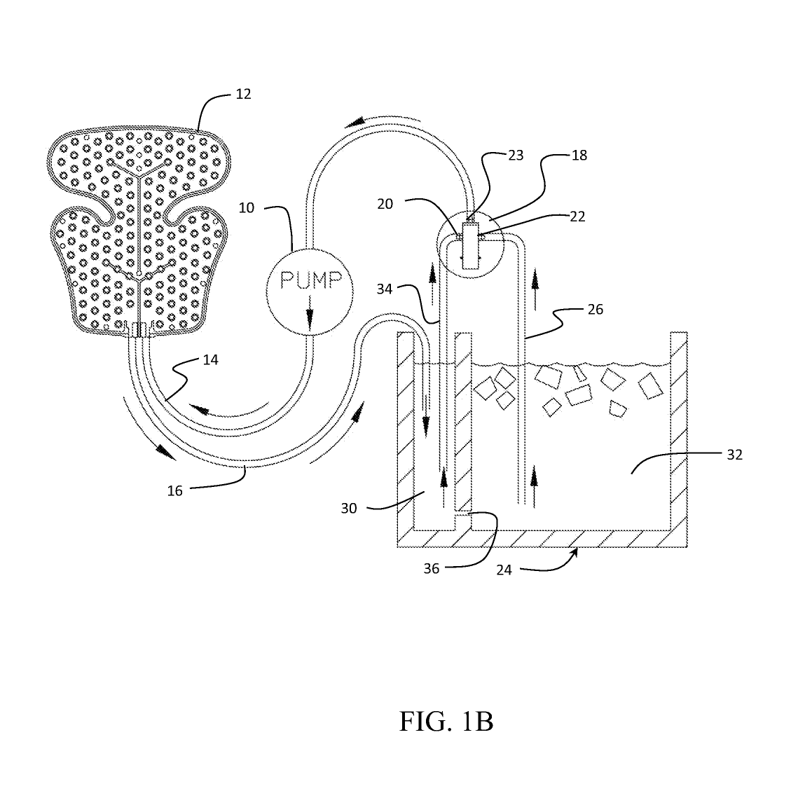

Referring to the drawings, FIG. 1A shows a first exemplary embodiment of the elements of the system. Water is provided through a pump 10 entering a pad 12 through an inlet line 14. The flow of water is much faster than prior art cold therapy designs so that water entering the pad 12 is close to the same temperature as the water leaving the pad at outlet line 16. The pump 10 pulls water through a thermo-mechanical mixing valve 18, to be described in greater detail subsequently, having a first input port 20 and a second inlet port 22 with the pump connected to an outlet port 23. The first inlet port 20 receives water that has circulated through the pad 12 and exited in outlet line 16. The second inlet port 22 of the thermo-mechanical valve receives water from a reservoir 24 through a cold conduit 26. The reservoir contains a cold fluid, typically ice water or other cooled or refrigerated fluid, to provide the desired cooling to the pad 12. For description of the embodiments herein water will be the example fluid.

In the first embodiment, the connection between the first input port and outlet line 16 is direct through first bifurcated line 28a and a second bifurcated line 28b is provided to recirculate water to the reservoir 24 for specific flow conditions to be described subsequently. In a second embodiment shown in FIG. 1B, the reservoir 24 incorporates a holding reservoir 30 which receives returning water from the outlet line 16 and a cold reservoir 32. A warm conduit 34 replaces the bifurcated lines 28a and 28b to provide water from the holding reservoir to the first inlet port 20. The cold reservoir 32 is provided for holding the ice water and is connected to second inlet port 22 in the thermo-mechanical mixing valve through cold conduit 26. A leveling port 36 may be employed to equilibrate the water levels between the holding reservoir 30 and cold reservoir 32.

Initially when the pad 12 is first applied to a patient and the pump 10 is turned on, the water pumped through the thermo-mechanical mixing valve 18 will be drawn from the cold reservoir 32 and into the pad which will cool to a little above freezing (or the refrigerated temperature of the water in the cold reservoir). Because the water is below a lower temperature threshold, the thermo-mechanical mixing valve will substantially close off the second inlet port 22 receiving the iced water and only recirculate the water from the pad outlet line 16, directly from the pad outlet line for the first embodiment or from the holding reservoir 30 for the second embodiment, through inlet port 20 and through the pump 10 to the pad 12 until the water reaches the predetermined safe temperature. At that point the thermo-mechanical mixing valve 18 will adjust to allow inflow through cold conduit 26 to the second inlet port 22 and allow some iced water to enter the system to maintain that preset temperature. With a large pad such as a shoulder pad, the unit may have a hard time keeping up with the amount of heat the body is adding to the system and the thermo-mechanical mixing valve may shift to close the first inlet port 20 upon reaching a high threshold temperature resulting in a higher percentage of iced water flowing through the pad 12. In any case, no matter what size pad, where it is placed on the body, or the health of the patient's circulation, the water being fed to the pad 12 will always be adjusted automatically to remain in a safe range.

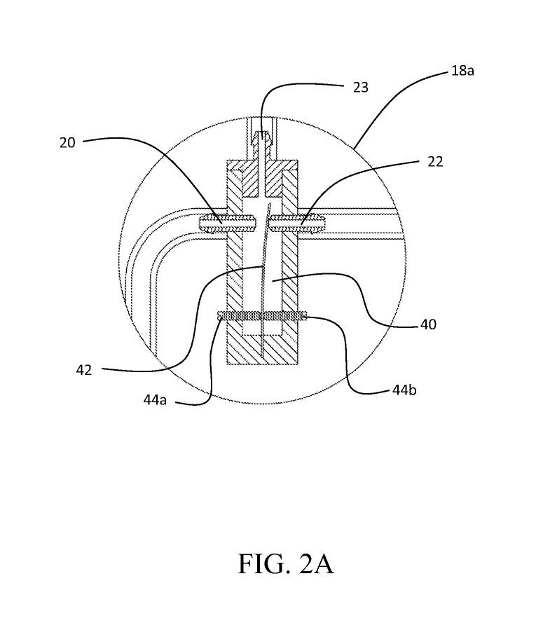

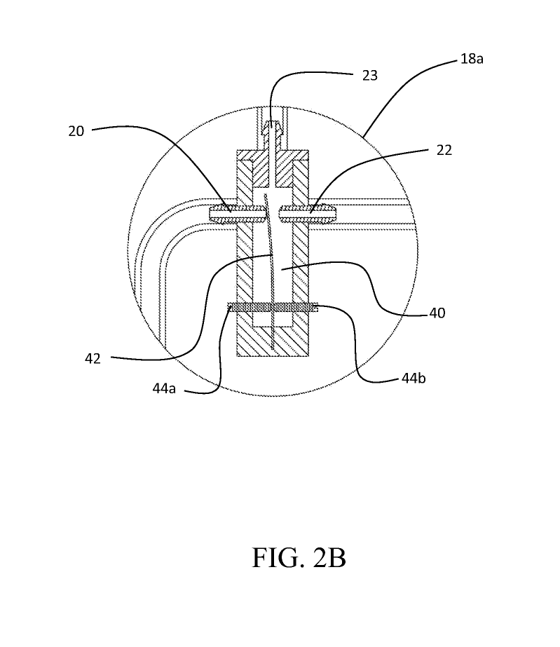

The thermo-mechanical mixing valve 18 operates automatically for water temperature adjustment. Water flowing through the valve is in contact with an adjustment element which is reactive to the temperature to which it is exposed and thus alters shape based on the temperature of the water. In a first example a bimetal valve 18a may be employed as the thermo-mechanical valve by the embodiments disclosed herein. FIG. 2A shows a sealed chamber 40 with the two inlet ports on opposite sides of the chamber, the second inlet port 22 receiving cold fluid from the reservoir and the first inlet port 20 receiving warmer fluid from the outlet port of the treatment pad. A bimetal strip 42 reactive by bending in accordance with exposure temperature is oriented in the chamber to block off the second inlet port 22, as shown in FIG. 2A, when the temperature of the fluid in the chamber is below a predetermined lower threshold or the first inlet port 20, as shown in FIG. 2B, when the treatment fluid in the chamber is above a predetermined upper threshold. Adjustment screws 44 calibrate the temperature at which the fluid in the chamber is maintained. The outlet port 23 delivers the mixed treatment fluid from the chamber 40 though the pump 10 and on to the inlet line 14 of the treatment pad 12. The bimetal strip will typically be made of copper on one surface and stainless steel on the other. In an exemplary embodiment, a bimetal strip of about 2.5″ in length and 0.040 inches in thickness is employed. Most of the time during operation neither port will be closed completely. The thermo-mechanical mixing valve will typically find an equilibrium where each of the ports is delivering the proper percentage to maintain the correct temperature. Typically during the operation of the unit, there are only two times when the valve will be at either of the two extremes. The first is at start up when there is no water coming back from the pad. What will happen is the valve will close the ice water inlet, since the water will be near freezing, which will cause water to be drawn from the reservoir backwards up the return tube and into the mixing valve through the return inlet port. Once the pad is full and water is returning the valve will operate as intended. The only other time the valve will be at an extreme is when the ice in the reservoir melts and the mixing valve will pull only from the reservoir.

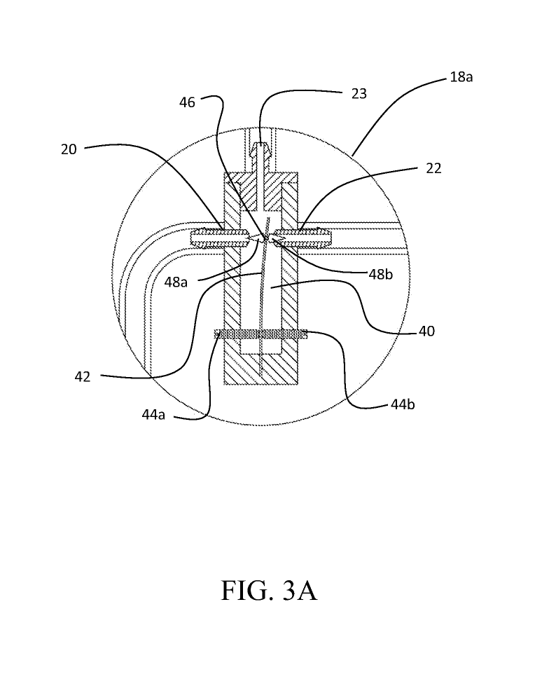

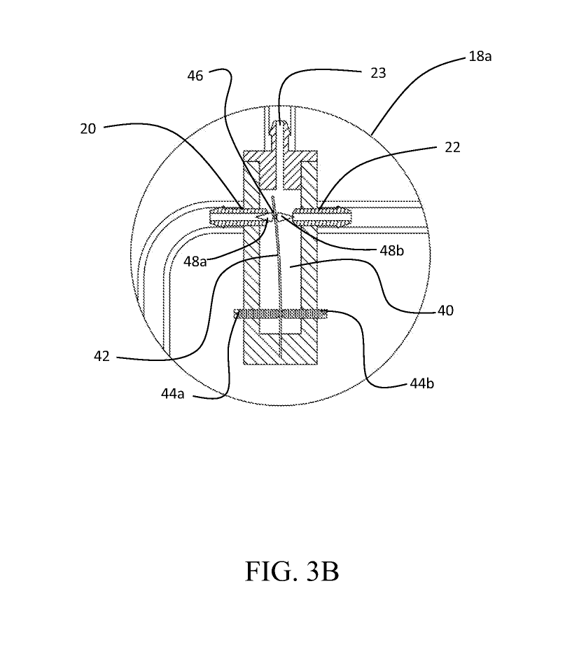

Greater control of the flow rates of water entering the sealed chamber of the bimetal valve can be accomplished as shown in FIG. 3A where a dual ended tapered needle valve 46 is adapted for a first end 48a to be received in in the first inlet port 20 and a second end 48b to be received in the second inlet port 22 to gradually increase or decrease the flow of fluid as the needle valve is retracted from or inserted into each of the ports. The bimetal strip 42 is oriented in the chamber and attached to the dual ended tapered needle valve to act on the dual ended needle valve causing the respective ends to be inserted or retracted from each of the ports depending on the temperature of the fluid in the chamber, causing a restriction of flow in the second inlet port 22, as shown in FIG. 3A, when the fluid in the chamber is below a predetermined lower threshold temperature or in the first inlet port 20, as shown in FIG. 3B, when the treatment fluid in the chamber is above a predetermined upper threshold temperature. Flexing of the bimetal strip 42 across the range from one extremity of motion to the opposite extremity of motion reactive to the temperatures between the lower and upper thresholds allows smooth control of flow mixing between the first and second inlet ports. Sizing of the pump, inlet and outlet ports and lines of the system are desirable for a flow of about 8 oz/min.

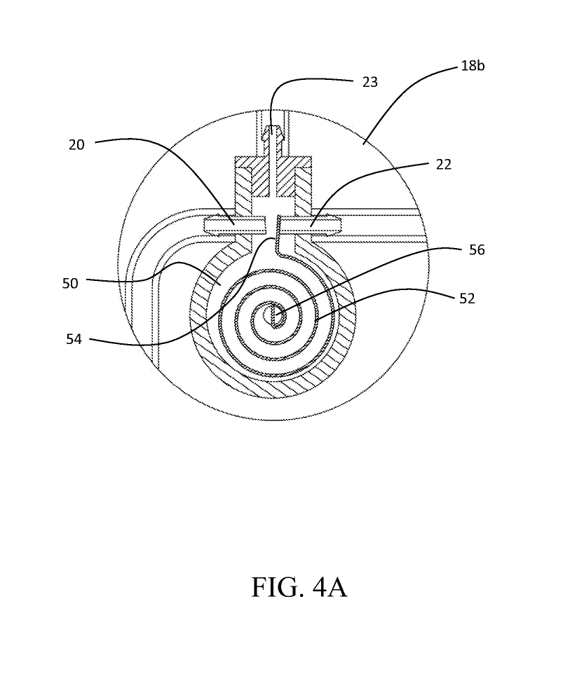

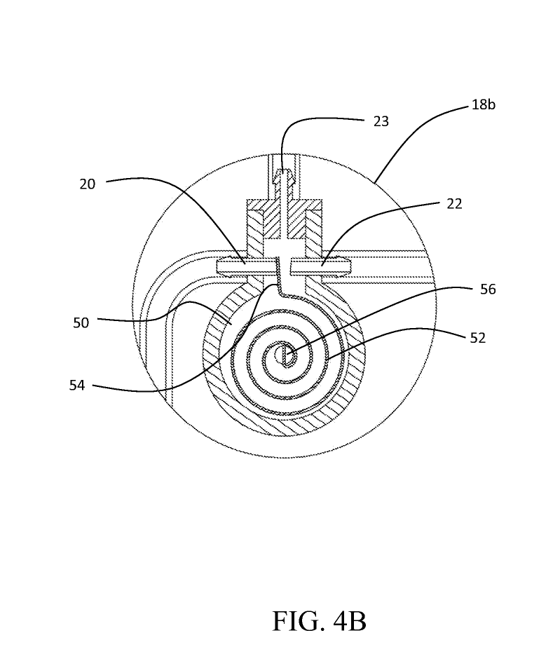

Alternatively, as shown in FIG. 4A, the mixing valve 18b employs the sealed chamber 50, which may be substantially circular, connected to the two inlet ports on opposite sides of the chamber and a bimetal coil 52 which is reactive by winding or unwinding in accordance with exposure temperature is oriented in the chamber to block off the second inlet port 22 with an extending tang 54, as shown in FIG. 4A, when the fluid in the chamber is below a predetermined lower threshold temperature or the first inlet port 20, as shown in FIG. 4B, when the treatment fluid in the chamber is above a predetermined upper threshold temperature. An adjustment screw 56 at the center of the bimetal coil is employed to calibrate the temperature at which the fluid in the chamber is maintained. In an exemplary embodiment, a bimetal coil of 1.25″ diameter, 0.35″ width and 0.040″ thickness is employed.

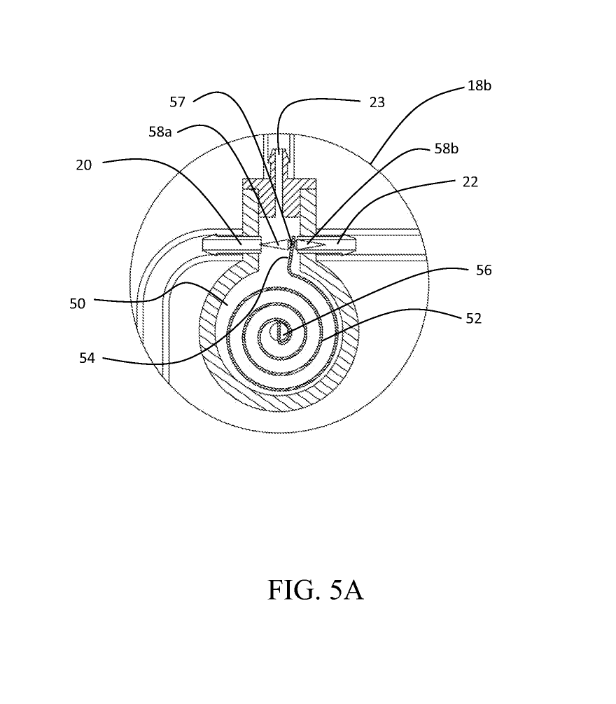

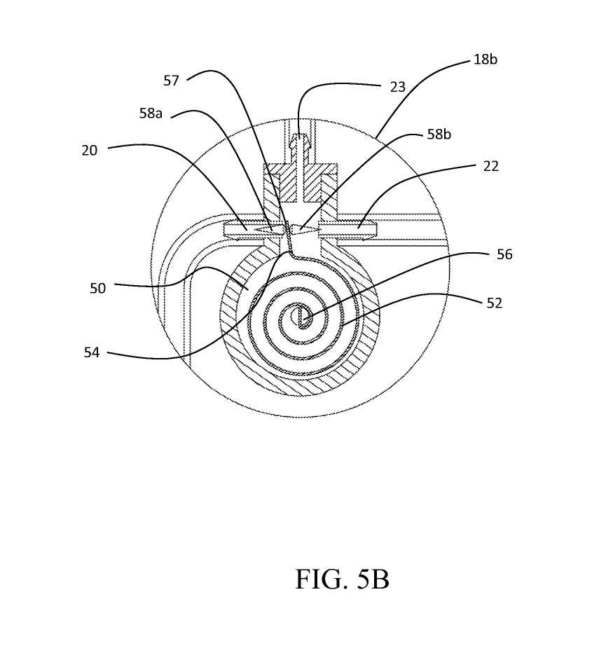

As with the bimetal strip, greater control of flow can be achieved with the bimetal coil 52 as shown in FIG. 5A where a dual ended tapered needle valve 57 is connected to the tang 54 and adapted for a first end 58a to be received in in the first inlet port 20 and a second end 58b to be received in the second inlet port 22 to gradually increase or decrease the flow of fluid as the needle valve is retracted from or inserted into each of the ports. The bimetal coil is oriented in the chamber and attached to the dual ended tapered needle valve to act on the dual ended needle valve causing the respective ends to be inserted or retracted from each of the ports depending on the temperature of the fluid in the chamber, causing a restriction of flow in the second inlet port, as shown in FIG. 5A, when the fluid in the chamber is below a predetermined lower threshold temperature or in the first inlet port, as shown in FIG. 5B, when the treatment fluid in the chamber is above a predetermined upper threshold temperature. As with the bimetal strip, winding and unwinding of the bimetal coil across the range from one extremity of motion to the opposite extremity of motion reactive to the temperatures between the high and low thresholds allows smooth control of flow mixing between the first and second inlet ports.

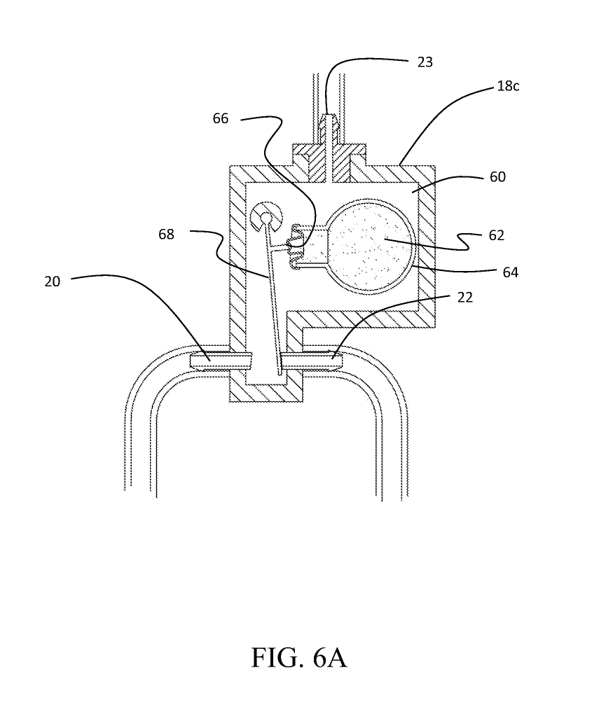

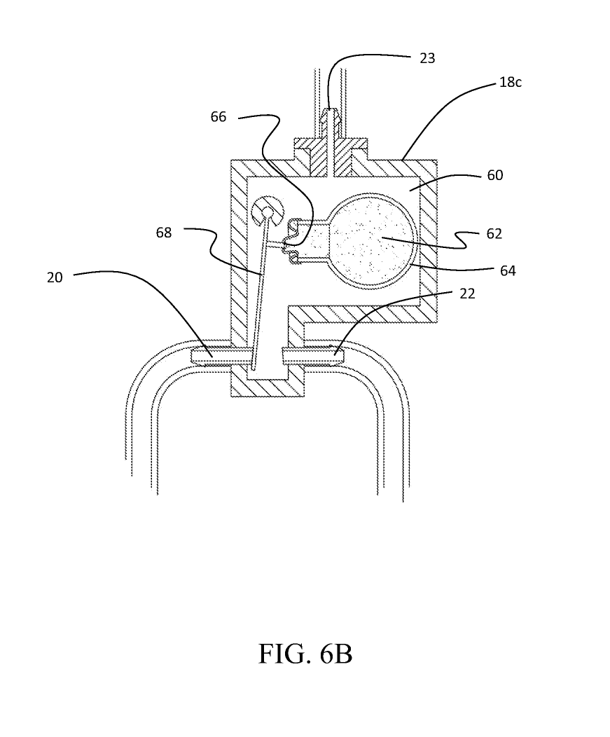

In yet another alternative embodiment, as shown in FIG. 6A, the thermo-mechanical mixing valve is a pressure valve 18c which employs a sealed chamber 60, connected to the two inlet ports on opposite sides of the chamber and a thermo-mechanical actuation element 62 which is reactive providing expansion or contraction of a gas, liquid or gel that is temperature sensitive contained in a vessel 64 supported within the chamber. A diaphragm (or piston) 66 is attached to a lever 68 which is laterally displaced by the fluid in actuation element 62 moving diaphragm 66 between a first position substantially sealing the second inlet port 22, shown in FIG. 6A, when the fluid in the chamber 60 is below a predetermined lower threshold temperature and to a second position substantially sealing the first inlet port 20, as shown in FIG. 6B, when the treatment fluid in the chamber is above a predetermined upper threshold temperature.

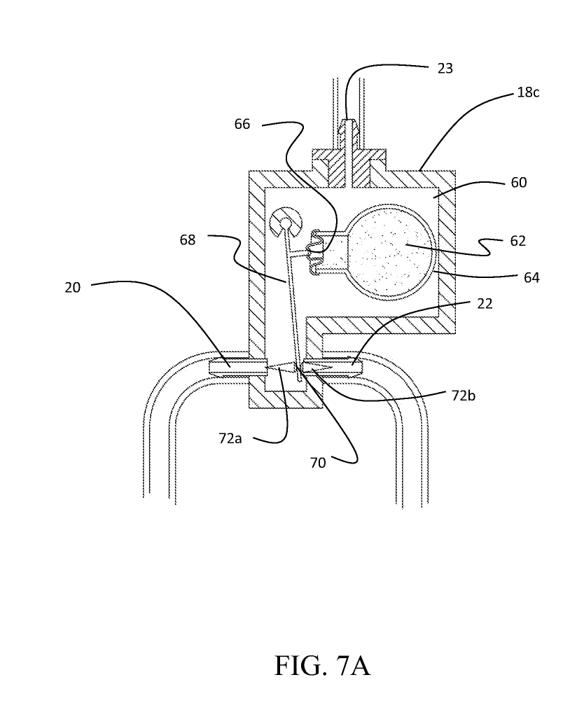

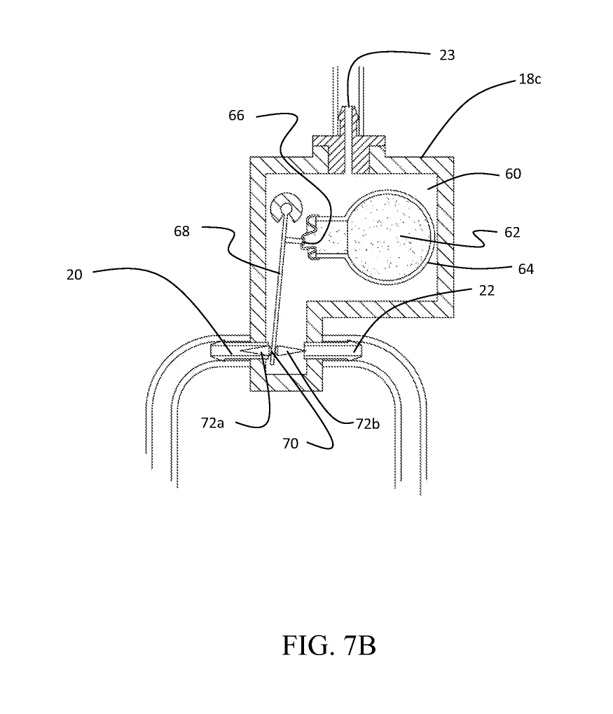

As with the bimetal mixing valves, greater control of flow can be achieved with the pressure valve as shown in FIG. 7A where a dual ended tapered needle valve 70 is connected to the lever 68 and adapted for a first end 72a to be received in in the first inlet port 20 and a second end 72b to be received in the second inlet port 22 to gradually increase or decrease the flow of fluid as the needle valve is retracted from or inserted into each of the ports. The lever 68 is oriented in the chamber and attached to the dual ended tapered needle valve to act on the dual ended needle valve causing the respective ends to be inserted or retracted from each of the ports depending on the temperature of the fluid in the chamber, causing a restriction of flow in the second inlet port, as shown in FIG. 7A, when the fluid in the chamber is below a predetermined lower threshold temperature or in the first inlet port, as shown in FIG. 5B, when the treatment fluid in the chamber is above a predetermined upper threshold temperature. As with the bimetal mixing valves, expansion and contraction of the fluid contained in the actuation element 62 across the range from full contraction to full expansion reactive to the temperatures between the high and low thresholds allows smooth control of flow mixing between the first and second inlet ports.

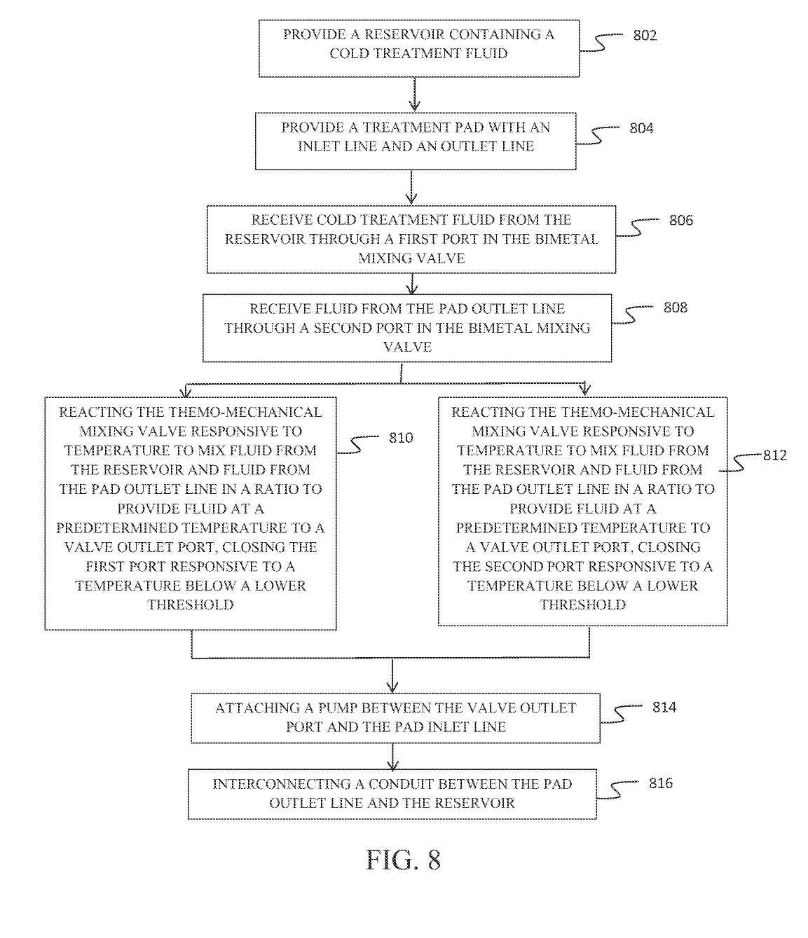

The embodiments disclosed herein allow a method for operation of a cold therapy pad as shown in FIG. 8. A reservoir is provided containing a cold treatment fluid, step 802, and a treatment pad is provided with an inlet line and an outlet line, step 804. A thermo-mechanical mixing valve receives cold treatment fluid from the reservoir through a first port, step 806, and fluid from the pad outlet line through a second port, step 808. The thermo-mechanical mixing valve is reactive responsive to temperature to mix fluid from the reservoir and fluid from the pad outlet line in a ratio to provide fluid at a predetermined temperature to a valve outlet port, closing the first port responsive to a temperature below a lower threshold, step 810 and closing the second port responsive to a temperature above an upper threshold, step 812. The reaction of the thermo-mechanical mixing valve may be accomplished by bending of the bimetal strip, winding or unwinding of the bimetal coil or expanding or contracting a fluid in a sealed vessel in the embodiments as described. A pump is attached between the valve outlet port and the pad inlet line, step 814, and a conduit is interconnected between the pad outlet line and the reservoir, step 816.

Having now described various embodiments of the disclosure in detail as required by the patent statutes, those skilled in the art will recognize modifications and substitutions to the specific embodiments disclosed herein. Such modifications are within the scope and intent of the present disclosure as defined in the following claims.

What is claimed is:

1. A cold therapy unit comprising:
a reservoir containing a cold treatment fluid;
a treatment pad with an inlet line and an outlet line;
a thermo-mechanical mixing valve receiving cold treatment fluid from the reservoir through a first inlet port and fluid from the pad outlet line through a second inlet port, said thermo-mechanical mixing valve reactive responsive to temperature to mix fluid from the reservoir and fluid from the pad outlet line in a ratio to provide fluid at a predetermined temperature to a valve outlet port;
a pump attached to the valve outlet port and the pad inlet line to deliver the mixed treatment fluid from the mixing valve to the pad; and
a conduit interconnecting the pad outlet line and the reservoir.

2. The cold therapy unit as defined in claim 1 further comprising a first bifurcated line connected between the pad outlet line and the second inlet port and a second bifurcated line as the conduit to recirculate water to the reservoir as the conduit.

3. The cold therapy unit as defined in claim 1 wherein the reservoir comprises:
a cold reservoir connected to the first port; and,
a holding reservoir connected to the conduit from the pad outlet line, said holding reservoir connected to the second inlet port.

4. The cold therapy unit as defined in claim 3 wherein the cold reservoir and holding reservoir are interconnected with a leveling port.

5. The cold therapy unit as defined in claim 1 wherein the thermo-mechanical mixing valve comprises a bimetal mixing valve.

6. The cold therapy unit as defined in claim 5 wherein the bimetal mixing valve comprises:
a sealed chamber with the first and second inlet ports on opposite sides of the chamber, the first inlet port receiving cold fluid from the reservoir and the second inlet port receiving warmer fluid from the outlet line of the treatment pad; and,
a bimetal strip reactive by bending in accordance with exposure temperature is oriented in the chamber to block off the first inlet port when the temperature of the fluid in the chamber is below a predetermined lower threshold or the second inlet port when the treatment fluid in the chamber is above a predetermined upper threshold.

7. The cold therapy unit as defined in claim 6 further comprising adjustment screws engaged to the bimetal strip to calibrate temperature at which the fluid in the chamber is maintained.

8. The cold therapy unit as defined in claim 6 further comprising:
a dual ended tapered needle valve having
a first end received in the first inlet port and
a second end received in the second inlet port, said first and second ends adapted to gradually increase or decrease a flow of fluid associated inlet port as the needle valve is retracted from or inserted into each of the ports,
said bimetal strip oriented in the chamber to act on the dual ended needle valve causing the respective ends to be inserted or retracted from the associated ports depending on the temperature of the fluid in the chamber causing a restriction of flow in the first inlet port when the fluid in the chamber is below a predetermined threshold temperature or in the second inlet port when the treatment fluid in the chamber is above a predetermined threshold temperature.

9. The cold therapy unit as defined in claim 5 wherein the bimetal mixing valve comprises:
a sealed chamber with first and second inlet ports on opposite sides of the chamber, the first inlet port receiving cold fluid from the reservoir and the second inlet port receiving warmer fluid from the outlet line of the treatment pad;
a bimetal coil reactive by winding or unwinding in accordance with exposure temperature oriented in the chamber to block off the first inlet port when the temperature of the fluid in the chamber is below a predetermined lower threshold or the second inlet port when the treatment fluid in the chamber is above a predetermined upper threshold.

10. The cold therapy unit as defined in claim 9 further comprising an adjustment screw at the center of the bimetal coil to calibrate temperature at which the fluid in the chamber is maintained.

11. The cold therapy unit as defined in claim 9 further comprising:
a dual ended tapered needle valve having
a first end received the first inlet port and
a second end received in the second inlet port, said first and second ends adapted to gradually increase or decrease a flow of fluid associated inlet port as the needle valve is retracted from or inserted into each of the ports,
said bimetal coil oriented in the chamber to act on the dual ended needle valve causing the respective ends to be inserted or retracted from the associated ports depending on the temperature of the fluid in the chamber causing a restriction of flow in the first inlet port when the fluid in the chamber is below a predetermined lower threshold temperature or in the second inlet port when the treatment fluid in the chamber is above a predetermined upper threshold temperature.

12. The cold therapy unit as defined in claim 1 wherein the thermo-mechanical mixing valve comprises a pressure mixing valve.

13. The cold therapy unit as defined in claim 12 pressure mixing valve comprises:
a sealed chamber with first and second inlet ports on opposite sides of the chamber, the first inlet port receiving cold fluid from the reservoir and the second inlet port receiving warmer fluid from the outlet line of the treatment pad;
a thermo-mechanical actuation element reactive to temperature by providing expansion or contraction of a fluid that is temperature sensitive contained in a vessel supported within the chamber, a diaphragm is attached to a lever which is laterally displaced by the fluid in the actuation element moving the lever between a first position substantially sealing the second inlet port when the fluid in the chamber is below a predetermined lower threshold temperature and to a second position substantially sealing the first inlet port when the treatment fluid in the chamber is above a predetermined upper threshold temperature.

14. The cold therapy unit as defined in claim 13 further comprising:
a dual ended tapered needle valve having
a first end received the first inlet port and
a second end received in the second inlet port, said first and second ends adapted to gradually increase or decrease a flow of fluid associated inlet port as the needle valve is retracted from or inserted into each of the ports,
said lever oriented in the chamber to act on the dual ended needle valve causing the respective ends to be inserted or retracted from the associated ports depending on the temperature of the fluid in the chamber causing a restriction of flow in the first inlet port when the fluid in the chamber is below a predetermined lower threshold temperature or in the second inlet port when the treatment fluid in the chamber is above a predetermined upper threshold temperature.

15. A method for providing cold therapy comprising:
providing a reservoir containing a cold treatment fluid;
providing a treatment pad with an inlet line and an outlet line;
connecting a bimetal mixing valve to receives cold treatment fluid from the reservoir through a first port and fluid from the pad outlet line through a second port;
closing the first port responsive to a temperature below a lower threshold by reaction of the thermo-mechanical mixing valve;
closing the second port responsive to a temperature above an upper threshold by reaction of the thermo-mechanical mixing valve;
attaching a pump between an outlet port on the thermo-mechanical mixing valve and the pad inlet line; and,
interconnecting a conduit between the pad outlet line and the reservoir.

16. The method for providing cold therapy as defined in claim 15 wherein the thermo-mechanical mixing valve incorporates a bimetal strip and said reaction of the bimetal mixing valve comprises bending of the strip.

17. The method for providing cold therapy as defined in claim 15 wherein the thermo-mechanical mixing valve incorporates a bimetal coil and said reaction of the thermo-mechanical mixing valve comprises winding or unwinding of the coil.

18. The method for providing cold therapy as defined in claim 15 wherein the thermo-mechanical mixing valve incorporates a fluid contained in a sealed vessel with a diaphragm or piston and said reaction of the thermo-mechanical mixing valve comprises the expansion or contraction of said fluid causing said diaphragm or piston to move out from or in towards said vessel.
